(19) United States
(12) Patent Application Publication
(10) Pub. No.: US 2025/0222668 A1
(43) Pub. Date: Jul. 10, 2025

(54) ACCOMMODATING INTRAOCULAR LENSES AND METHODS OF MAKING SAME

(71) Applicant: ForSight Vision6, Inc., South San Francisco, CA (US)

(72) Inventors: Guy Oren, South San Francisco, CA (US); Nicole Kahn-Dror, South San Francisco, CA (US); Rina Tsvet, South San Francisco, CA (US); Yael Gefen, South San Francisco, CA (US)

(21) Appl. No.: 18/850,305

(22) PCT Filed: Mar. 27, 2023
(86) PCT No.: PCT/US2023/016450

§ 371 Date: Sep. 24, 2024

Related U.S. Application Data
(60) Provisional application No. 63/324,542, filed on Mar. 28, 2022.

(51) Int. Cl. B29D 11/00; A61F 2/16; B29D 11/02

(52) U.S. Cl. CPC B29D 11/0073; A61F 2/16; B29D 11/023; A61F 2002/1689

(57) ABSTRACT

A method of manufacturing an accommodating intraocular lens including molding an optical component having at least a central portion formed of an optically clear silicone material; molding a second component, the second component comprising a silicone material; activating surfaces of the optical component and surfaces of the second component in a plasma chamber; and covalently bonding together without adhesive the optical component and the second component to form a lens capsule having an internal chamber.

### CROSS-REFERENCE TO RELATED APPLICATIONS

This application claims priority to co-pending U.S. Provisional Patent Application Ser. No. 63/324,542, filed Mar. 28, 2022, the disclosure of which is hereby incorporated by reference in its entirety for all purposes.

### BACKGROUND

IOLs are typically implanted after cataract extractions. Generally, IOLs are made of a foldable material, such as silicone or acrylics, for minimizing the incision size and improving patient recovery time. Most commonly used IOLs are single-element lenses that provide a single focal distance for distance vision. Accommodating intraocular lenses (AIOLs) have also been developed to provide adjustable focal distances (or accommodations) that rely on the natural focusing ability of the eye, for example, as described in US 8,414,646, U.S. Pat. Nos. 8,167,941, 9,913,712, 10,258,805, and US 2019/0269500, which are each incorporated by reference herein in their entireties.

AIOLs are beneficial for patients not suffering from cataracts, but who wish to reduce their dependency on glasses and contacts to correct their myopia, hyperopia and presbyopia. Intraocular lenses used to correct large errors in myopic, hyperopic, and astigmatic eye are called “phakic intraocular lenses” and are implanted without removing the crystalline lens. In some cases, aphakic IOLs (not phakic IOLs) are implanted via lens extraction and replacement surgery even if no cataract exists. During this surgery, the crystalline lens is extracted and an IOL replaces it in a process that is very similar to cataract surgery. Refractive lens exchange, like cataract surgery, involves lens replacement, requires making a small incision in the eye for lens insertion, use of local anesthesia and lasts approximately 30 minutes.

IOLs, particularly AIOLs, may incorporate liquids in fluid chambers such that accommodation is achieved with the help of fluid-actuated mechanisms. A force exerted on a portion of the lens is transmitted via the fluid to deform a flexible layer of the lens resulting in accommodative shape change of the IOL. For example, ciliary muscle movements of the eye may be harnessed by components of an AIOL to drive shape change and accommodation. The AIOLs can achieve an optical power or diopter (D) in a desired range due to shape change of the optic upon application of a small amount of force (e.g., as little as 0.1-1.0 grams force (gf)) applied by the eye tissue. The AIOLs provide reliable dioptric change by harnessing small forces. A chamber for containing liquid materials that is formed by flexible layers of elastomeric material can change shape and thus, power of the lens depending on the volume of liquid. As fill volume increases beyond the chamber volume, the flexible layers can bulge outward creating a lens with a greater focal length.

There is need in the art for improved manufacturing of shape changing lenses that provide improved properties for patients in need. The disclosure is directed to this, as well as other, important ends.

### SUMMARY

In an aspect, described is a method of manufacturing an accommodating intraocular lens including molding an optical component having at least a central portion formed of an optically clear silicone material; molding a second component, the second component comprising a silicone material; activating surfaces of the optical component and surfaces of the second component in a plasma chamber; and covalently bonding together without adhesive the optical component and the second component to form a lens capsule having an internal chamber.

The method can further include applying a vacuum pressure within the plasma chamber during the activating. The vacuum pressure can be between 0.01 mbar and 1 mbar. A process power of the plasma chamber during the activating can be between 270-300 W. The method can further include supplying a gas to the plasma chamber during the activating. The gas can be Argon, atmospheric air, or oxygen. The gas can be 70%-100% oxygen. The gas can be supplied with or without applying vacuum pressure to the plasma chamber.

The method can further include holding the optical component and the second component within a fixture during the covalently bonding. The fixture can be designed to apply an amount of compression between the surfaces of the optical component and the surfaces of the second component. The amount of compression is between 30-100 gf. The fixture can be designed to hold the optical component and the second component in contact with one another without applying compressive force on the optical component and the second component. The fixture can be formed of a material that does not bond to the optical component or the second component during covalently bonding. The fixture can be a high-heat resistant material. The fixture can be polytetrafluoroethylene (PTFE). The fixture can include a first surface contacting the optical component and a second surface contacting the second component. The first surface and the second surface can be modified to prevent bonding to optical component and the second component, respectively. The first surface and the second surface can be modified by coating or by laser texturing. The first surface can be on a fixture lid and the second surface can be on a fixture base, wherein the fixture lid and the fixture base are configured to mate with one another while also holding the optical component and the second component relative to one another. The fixture lid can include a pin and the fixture base comprises a helical groove, wherein the pin rides within the helical groove as the fixture lid is locked with the fixture base. The fixture can be spring-loaded and include a spring inside the fixture lid configured to apply a load onto the optical component toward the second component. The load applied can be between 0.1-1.0 kg force.

The optically clear silicone material of the central portion can have a durometer of Shore A 10 to Shore A 90. A transparency for the optically clear silicone material of the optical component can be greater than 85% at 550 nm. The optical component can further include a material displacement component comprising a silicone material. Molding an optical component can include molding as a monolithic capsule the material displacement component and the central portion. Molding an optical component can include molding separately the optical component and the material displacement component. The silicone material of the material displacement component can be configured to reduce edge glare and straylight and can have a high elongation and tear strength. The Shore hardness of the silicone material of the material displacement component can be between Shore A 10 and Shore A 90. T transparency for the silicone material of the material displacement component can be less than 85% at 550 nm. The material displacement component can be arranged at a periphery located outside a visual zone of the intraocular lens and the central portion can be arranged at a central region within the visual zone of the intraocular lens. The material displacement component can include at least one force transfer arm coupled to a perimeter ring. The optically clear silicone material of the central portion and the silicone material of the material displacement component can include poly(dimethylsiloxane) (PDMS). The silicone material of the material displacement component can be opacified or made translucent. The silicone material of the material displacement component can include a high filler content, a pigment, or a surface texture. The second component can be a posterior lens. The posterior lens can be an optically clear material. The optically clear material of the posterior lens can have a Shore hardness that is between Shore A 10 and Shore A 90.

The method can further include molding a haptic and covalently bonding the haptic to the lens capsule using plasma bonding without adhesive. The haptic can be a silicone material having a Shore A hardness between Shore A 50 and Shore A 80. The method can further include sealing the internal chamber and injecting an optically clear liquid into the internal chamber. The optically clear liquid can be silicone oil or fluorosilicone oil.

### BRIEF DESCRIPTION OF THE DRAWINGS

These and other aspects will now be described in detail with reference to the following drawings. Generally, the figures are exemplary and are not to scale in absolute terms or comparatively but are intended to be illustrative. Relative placement of features and elements is modified for the purpose of illustrative clarity.

FIG. 1A is a perspective view of an intraocular lens;

FIGS. 1B and 1C are cross-sectional view of the lens of FIG. 1A taken along lines B-B and C-C, respectively;

FIGS. 2A-2B are perspective top and bottom views, respectively, of an anterior lens capsule of an intraocular lens molded as a unitary piece;

FIGS. 2C-2D are perspective top and bottom views, respectively, of an anterior lens capsule of an intraocular lens molded as two component parts;

FIG. 2E is a perspective view of a posterior lens of an intraocular lens molded as a unitary piece;

FIG. 2F is a perspective view of a posterior lens of an intraocular lens molded as two component parts;

FIGS. 2G-2H are perspective top and bottom views, respectively, of a posterior lens coupled to a supporting perimeter ring of an intraocular lens;

FIG. 3 is an overview of an implementation of a manufacturing process for creating an intraocular lens;

FIGS. 4A-4B is a detailed overview of a manufacturing process for creating an intraocular lens;

FIGS. 5A-5B illustrate portions of an assembly fixture for plasma processing of intraocular lens component parts;

FIGS. 5C-5E illustrate the lid of the assembly fixture of FIG. 5B;

FIG. 5F is a cross-sectional view of the lid taken along line A-A of FIG. 5C;

FIG. 5G shows the fixture of FIGS. 5A-5B arranged for coupling to one another;

FIG. 5H is a cross-sectional view of the assembly fixture;

FIG. 6A is another implementation of an assembly fixture for plasma processing of intraocular lens component parts;

FIG. 6B is a top-down view of the assembly fixture of FIG. 6A;

FIG. 6C is a cross-sectional view of the assembly fixture of FIG. 6A;

FIGS. 7A-7B are cross-sectional views of an implementation of an intraocular lens showing the tapered shape of the force transfer arms; and

FIGS. 7C-7D are cross-sectional views of another implementation of an intraocular lens.

### DETAILED DESCRIPTION

The optical elements of conventional IOLs are molded or machined components, generally produced from silicone or acrylic materials. The haptics of IOLs can either be incorporated into that initial molding/machining process, as in the case of a 1 piece IOL; or can be added as a secondary step in which the haptic is attached to the optic, as in the case of a 3 piece IOL. A new generation of “shape change” IOLs are constructed from sealed chambers filled with fluid that are capable of variable optical power in the eye following implantation. Securely forming a sealed chamber that can serve as a component in an AIOL is a critical aspect of constructing a shape change AIOL that is safe for human use. To date, the process of forming the sealed chamber of AIOLs involves molding of silicone or acrylic parts and bonding them together using an adhesive. In the process of forming a shape change AIOL, there may be a single adhesion process or there may be multiple adhesion processes to join various AIOL components. A single adhesion step can be utilized if multiple functional elements of the AIOL are molded together as an integrated “monolithic” components. Molding accommodating IOLs in a monolithic manner so that different regions of the IOL achieve their respective functions is challenging and restricts the material selection for optimizing device function. For example, the material displacement components can have different optical and mechanical requirements from the optic component. By molding these components together, a lens designer is restricted to using a single material for both components while an optimal lens might use different materials for each component. By separating the components, the peripheral material displacement components can be produced from non-optically clear materials that provide increased tensile strength, hardness, or tear strength compared to the material of the optical component. By separating these functional elements into individual components that are subsequently adhered, one can tailor material selections for the requirements of each functional component.

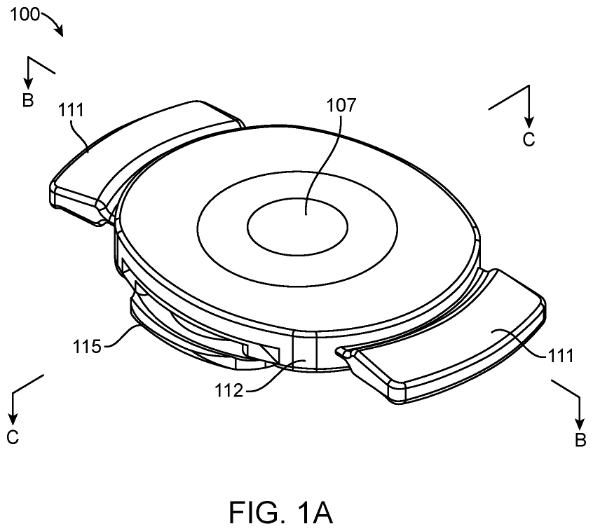

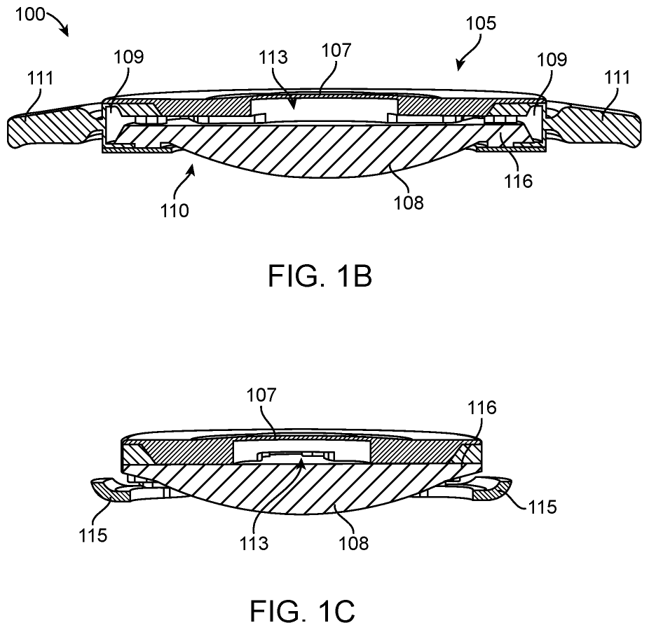

FIGS. 1A-1C illustrate an implementation of an accommodating intraocular lens 100. Generally, the lens 100 includes an anterior lens capsule 105, a posterior lens structure 110, a wing haptic 115. The anterior lens capsule 105 can include a dynamic anterior optic 107, peripheral membranes 109, and a pair of force transfer arms 111. The lens 100 includes an internal chamber 113 that is sealed and configured to hold a volume of liquid such as silicone oil. The force transfer arms 111 are arranged relative to the peripheral membrane 109 so that movements of the ciliary body are transferred by the arms 111 to compress the peripheral membrane 109 that urges the silicone oil within the chamber 113 to force the anterior optic 107 to bulge outward. Features of the lens 100 will be described in more detail below as well as in U.S. Pat. Nos. 8,414,646, 8,167,941, 9,913,712, 10,285,805, and U.S. 2019/0269500, which are each incorporated by reference herein in their entireties. The lenses are described to give context to the manufacturing procedures provided herein and are not intended to be limiting. Other silicone lens types are considered that have other structural features and achieve accommodation according to other mechanisms and may be produced according to the methods described herein.

The methods of manufacturing for intraocular lenses described herein allow for individually molding component parts having functionally disparate properties and bonding them together according to a process of plasma activation bonding without adhesives to create a lens having more accurate, quality optical properties. Surface activation prior to bonding achieves sufficiently high bonding strength without the need for any intermediate layer. For example, the non-optical material displacement component(s) can be molded separately from the optical components and then plasma bonded together. The design of molding components of the lens having functionally different properties and/or materials (e.g., optical and non-optical components) and then plasma bonding them together provides a simplicity to manufacturing. The manufacturing methods described herein achieve a simpler and more accurate mold design of the individual parts. This sort of design allows the tailoring of the materials properties of each component individually without the complexity of molding them as a unitary piece. An optical component formed of optically transparent materials and low shore hardness and/or tear strength can be molded separately from a non-optical component of the lens formed of translucent or opaque materials with a higher shore hardness and/or tear strength. The two components can then be bonded together without adhesive by incorporating a plasma bonding technique. This hybrid lens design allows for the tailoring of material properties of each component individually. The material displacement components can then have different optical and/or mechanical properties from the optic component. The peripheral structure outside the optical zone can be produced from non-optic material and reduce straylight and edge glare. The peripheral structure(s) can be produced from materials having high tensile strength, hardness, and/or tear strength as shown in Table 1 below. The mold design for each component individually is simpler and more accurate than attempting to mold an IOL in a monolithic fashion.

TABLE 1
Representative material properties of optical and
non-optical components of an anterior lens capsule

| | | Material Displacement |
|---|---|---|
| | Optical Component | Component |
| Hardness (Shore A) | 30-50 | 30-60 |
| Tear strength (ppi) | 25-50 | 80-200 |
| Transparency | Transparent | Translucent or opaque |
| | (>85% at 550 nm) | (<85% at 550 nm) |

The force transfer arms 111 and the perimeter ring 112 of the capsule can have a high elongation (>300%) and high tear force (>80 ppi up to about 200 ppi) and can be between 30-60 Shore A durometer (e.g., MED-6233, MED-4244, MED 5/4840, MED 5/4850) in order to achieve material displacement function. These components are at the perimeter of the lens and outside the visual zone and need not be optically clear. The wings 115, like the force transfer arms 111, need not be optically clear and are preferably opaque or translucent with a white colored pigment. The wings 115 can be between 50-80 Shore A durometer (e.g., MED-5/4880, MED-5/4870, MED-5/4860, MED-5/4850). The anterior optic 107, in contrast, must be optically clear. The posterior optic 108 of the posterior lens structure 110 must also be optically clear and preferably has a high refractive index (>1.43). The posterior optic 108 of the posterior lens structure 110 can be between 30-70, preferably about 30-50 Shore A durometer (e.g., MED-6820, MED1-6755) whereas the anterior optic 107 can be between 30-50 Shore A durometer (e.g., MED1-6755, MED-6233, MED-6820). The liquid filling the lens (e.g., 1000 cPs silicone or fluorosilicone oil) also must be optically clear. Straylight can be reduced by reducing the vertical surface area of internal features by creating an overall thinner lens (e.g., 6.8 mm³ vs. 5.9 mm³ if the anterior optic 107 height was reduced by 100 microns). An additional method to reduce edge glare is to opacify or frost (make translucent) the non-optical peripheral portion of the lens including the perimeter ring 112 and the force transfer arms 111. This can be achieved using PDMS material with high filler content, colored with pigment, or surface texturing. Reducing the lens thickness allows for smaller incision sizes and injection of the lens through an injector tip having small cross-section (i.e., <3.3 mm).

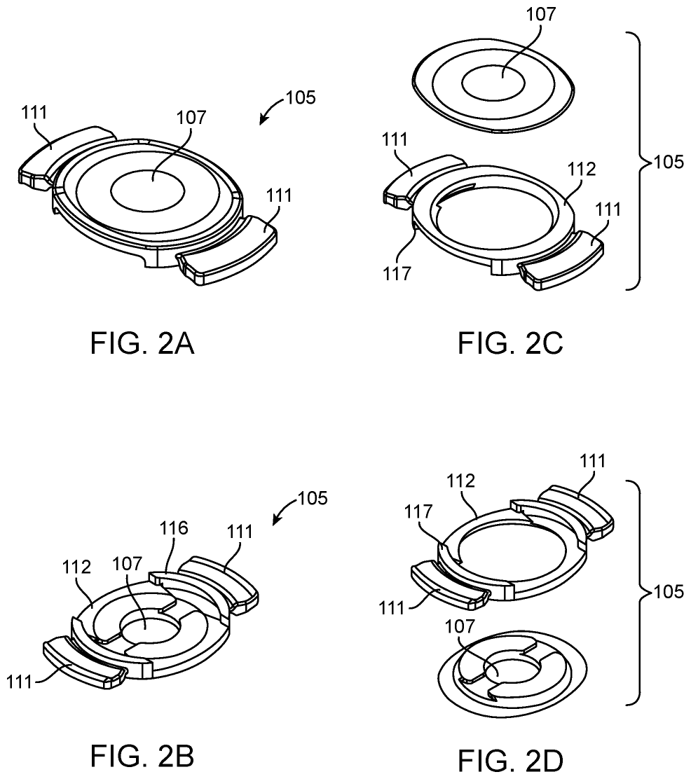

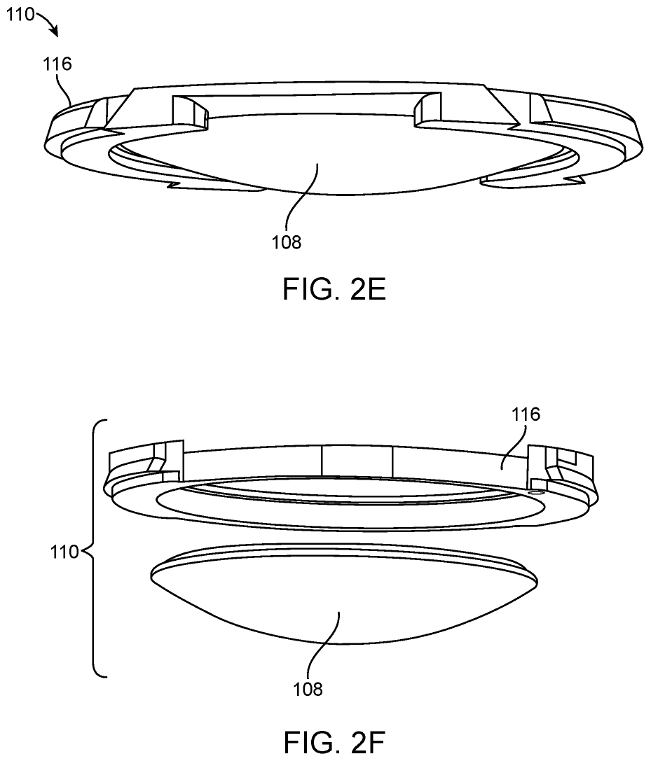

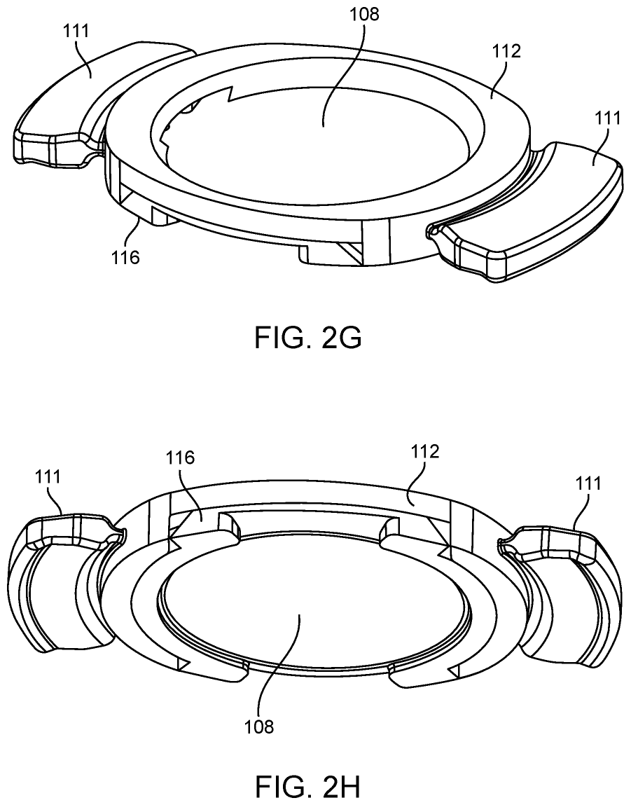

FIGS. 2A-2B show the anterior lens capsule 105 can be molded in a monolithic manner with the anterior optic 107 configured to undergo shape change for accommodation molded along with the non-optical components of the anterior lens capsule 105 such as the perimeter ring 112 with the peripheral membrane 109 and the force transfer arms 111. FIG. 2A is a top perspective view of the capsule 105 and FIG. 2B is a bottom perspective view of the capsule 105. FIGS. 2C-2D show the anterior lens capsule 105 can be molded in two parts with the anterior optic 107 molded separately from the non-optical components. The parts of the anterior lens capsule 105 can be plasma bonded together as described elsewhere herein prior to plasma bonding to the posterior lens structure 110 and wing haptic 115. FIG. 2C is a top perspective view of the capsule 105 and FIG. 2D is a bottom perspective view of the capsule 105.

The posterior lens structure 110 can also be molded in a monolithic manner with the posterior optic 108 molded together with a non-optical supportive perimeter region 116. The posterior lens structure 110 can also be molded in separate pieces where the optical and non-optical components are molded separately and bonded together as described elsewhere herein. FIG. 2E shows a monolithic posterior lens structure 110 with the optic 108 molded together with the non-optical supporting component 116 or rib edges. FIG. 2F shows the posterior lens structure 110 with the optic 108 molded separately from the non-optical supporting component 116. FIGS. 2G-2H show the non-optical components of the anterior capsule 105 coupled to the posterior lens structure 110. The non-optical supporting component 116 aligns and couples with the non-optical perimeter ring 112 of the anterior capsule 105.

Molding the multiple parts individually can reduce the variability in optical quality and eliminate the need for additional internal supports within one or more regions of the lens. The method allows for the anterior optic 107 of the lens capsule 105 (or posterior optic 108 of the posterior lens structure 110) to have more accurate thickness variation for improved optical quality. The method allows for the peripheral membranes 109 to be narrower to achieve a thinner AIOL overall. The molding in multiple parts can also provide easier release from the mold. It should be appreciated that one or more components of the IOL can be molded as a monolithic component or as two or more components molded individually and then bonded together as described herein. Any of a variety of combinations are considered.

The silicone parts are bonded together following exposure to plasma in a plasma chamber such as a cold plasma chamber (Atto plasma chamber, Diener, DE). In oxygen gas (O₂) plasma activation, PDMS (poly(dimethylsiloxane)) substrates are bombarded with ions and electrons, modifying the surface chemistry of the silicone components. The exposure of the parts to plasma in the plasma chamber creates active sites that can form chemically stable covalent bonds with other activated PDMS surfaces to form bridging Si—O—Si bonds at the interface. An alternate approach involves using Argon gas as a first step of the plasma activation to generate free radicals on the PDMS surface. Subsequent exposure to oxygen populates the surface with the active sites containing oxygen that can form the bridging Si—O—Si bonds. An alternate approach involves subsequent exposure to first Argon plasma and then oxygen plasma, or exposure to oxygen and argon plasma concurrently. Atmospheric air can be used in place of oxygen in any of the above scenarios. This plasma bonding allows one to form durable, fluid-tight bonds between optical components of an AIOL without the need of an adhesive. An adhesive-less process is particularly advantageous because adhesives between optical surfaces can reduce optical quality. To ensure a secure and uniform bond, pressure can be applied to the components being bonded. A uniform bond is critical to forming a safe device with sufficient optical quality.

The method of manufacturing an accommodating intraocular lens can include molding an optical component having at least a central portion formed of an optically clear silicone material and molding a second component that also comprises a silicone material. Surfaces of the optical component and the second component are activated in a plasma chamber and then covalently bonded together without adhesive, for example, to form a lens capsule having an internal chamber. The activating can be performed while applying a vacuum pressure within the plasma chamber or without applying a vacuum. Gas can be supplied to the plasma chamber during the activating including Argon, atmospheric air, or oxygen (70%-100%). The durometer of the silicone materials described herein can vary from Shore A 10 to Shore A 90 depending on desired structural characteristics of the component. For example, a central portion that is relatively large (e.g., 6 mm diameter) may be formed of a material having a higher Shore A durometer than a central portion that is smaller (e.g., 3 mm diameter). A posterior lens that is not configured to undergo shape change may also have a higher Shore A durometer range compared to a region of the lens that is configured to undergo shape change. Also, a component of the lens configured to provide support and/or fixation of the lens relative to the eye, such as a haptic, may have a Shore A durometer that is relatively harder than the central portion or a portion of the material displacement component, such as between Shore A 50 and Shore A 70.

The plasma activation parameters for materials of the anterior lens capsule 105 and posterior lens structure 110 to be bonded together (e.g., MED-6755 to MED-6820) as well as the anterior capsule-posterior lens assembly and the wing 115 to be bonded together (e.g., MED-6755/MED-6820 to MED-4870) can vary as described in more detail below. The vacuum pressure in the plasma chamber can be between 0.01 mbar and 1 mbar, or between 0.1 mbar and 0.5 mbar, or between about 0.2 mbar and 0.3 mbar. The gas supply or process of filling the empty plasma chamber with the gas can be atmospheric air and 80%-100% oxygen or Argon, 90%-100% oxygen or Argon, or 100% oxygen or Argon. The plasma chamber can be filled for 1-3 minutes prior to plasma processing. The exposure time for plasma activation of the surfaces can be between 10-60 seconds or between 15-50 seconds, or between 20-40 seconds, or between 25-30 seconds. In preferred embodiments, the exposure time for plasma activation of the surfaces is about 20, 25, 30, 35, 40, or up to about 45 seconds. The plasma processing power can be between about 270 W-300 W, between about 275 W-295 W, between about 280 W-290 W, or about 285 W. The plasma processing power can be between 70%-100% of 300 W, or between 80%-100% of 300 W, or between 90%-100% of 300 W. Table 2 below provides examples of the plasma activation parameters for treating MED-6755 and MED-6820 for bonding to each as well as for treating MED-6755/MED-6820 for bonding to MED-4870. In some implementations, bonding of harder materials such as MED-4870 to materials such as MED-6755 or MED-6820 may achieve efficient surface activation at a relatively high oxygen supply (e.g., 90%-100% O₂) and processing power (e.g., 95%-100% or 285 W-300 W), but over a relatively shorter process time (e.g., 20-30 seconds). In some implementations, atmospheric plasma can be used to activate the bonding surfaces. Atmospheric air can be used as the gas supply either with or without a pump to apply vacuum pressure to the plasma chamber.

The surface energy of the exposed parts is increased after exposure to plasma. The elevated surface energy of exposed parts decays with time once the parts are removed from the plasma chamber. At some point in this decay, the surface becomes insufficiently active for secure plasma bonding. For example, the parts may remain sufficiently active for up to 60 minutes following removal from the plasma chamber. The plasma activation of the surfaces remains active for a limited period of time after exposure, for example between 10-30 minutes. Surface activation of PDMS surfaces can decrease within 60 minutes after plasma exposure.

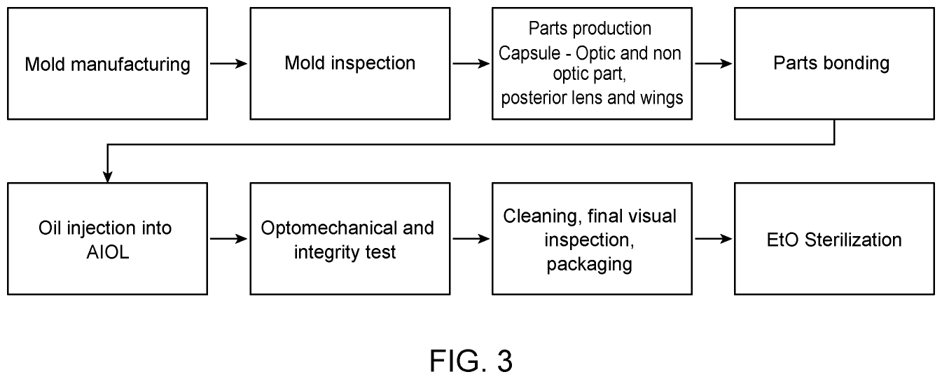

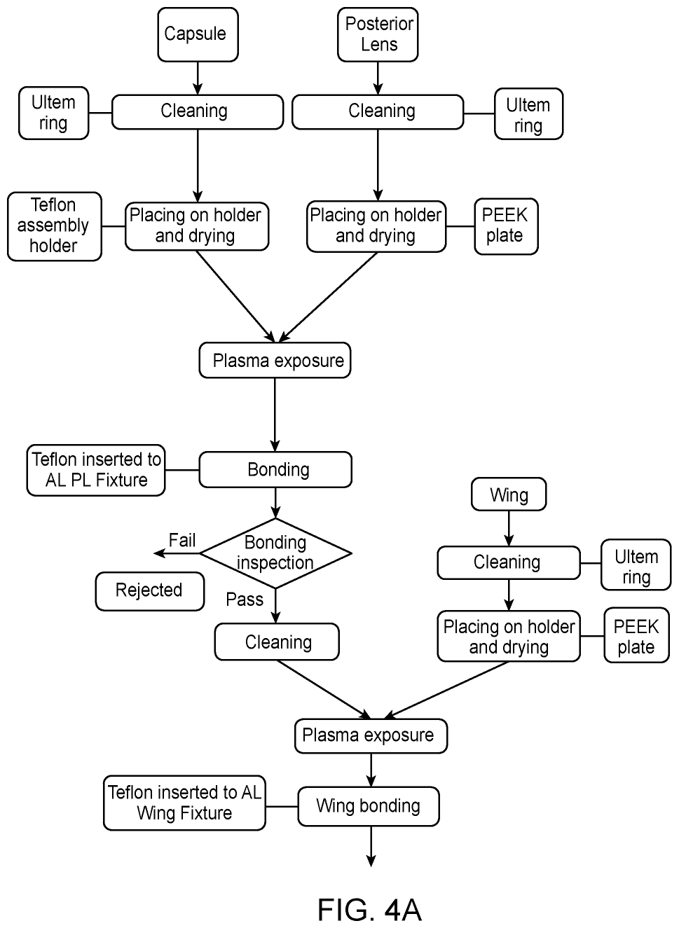

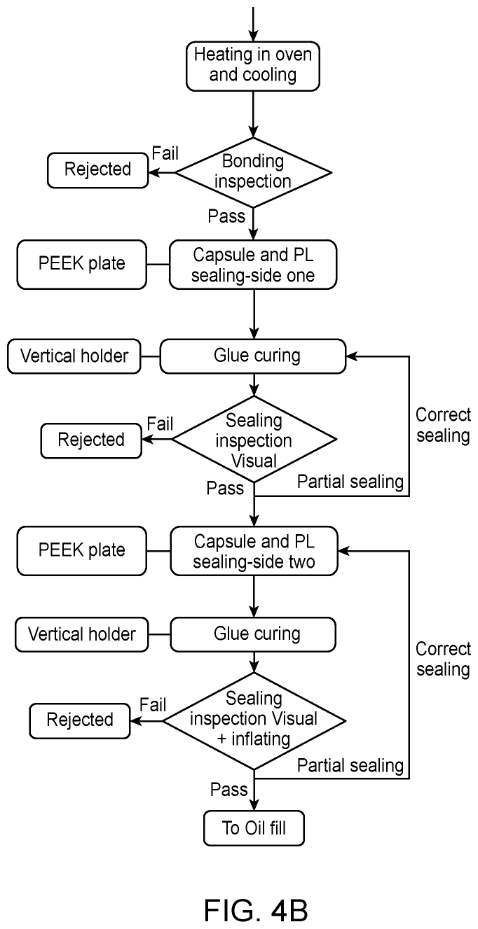

FIG. 3 illustrates an overview of an implementation of a manufacturing process of an intraocular lens, which generally includes steps of mold manufacturing, mold inspection, parts production, parts bonding forming AIOL, oil injection into AIOL, optomechanical and integrity testing, cleaning, final visual inspection, packaging, and sterilization. FIGS. 4A-4B is a more detailed view of an implementation of a manufacturing process of an intraocular lens. The molded components can vary including the anterior lens capsule 105, posterior lens structure 110 having the posterior optic 108, wing haptic 115, and any internal supports incorporated within the capsule 105. The combination of molded components for plasma bonding can vary as well. For example, the anterior lens capsule 105 and the posterior lens structure 110 can be plasma bonded together and then plasma bonded to the wing haptic 115. The components may be individually molded, cleaned, and dried prior to being assembled and undergoing plasma exposure. The components can be held within a fixture designed to apply an amount of compression (including no compression) during plasma bonding at a particular temperature and for a particular amount of time. Once all the component parts of the IOL are bonded to one another, the fluid chamber of the lens can then be sealed and filled with silicone oil. Each of the various steps of the plasma exposure and bonding processes are described in more detail below.

Various fixtures and holders are used to support the components during one or more steps in the manufacturing process. A fixture made of high-heat resistant material such as polyetherimide (e.g., ULTEM 1000), PEEK, or PTFE can be used during cleaning steps of the capsule, posterior lens, and wing. The holders and fixtures used during plasma treatments and bonding of the anterior lens capsule 105 and posterior lens structure 110 can be formed of PTFE (polytetrafluoroethylene). The posterior lens structure 110 and the wing 115 each can be positioned on a small plate formed of PEEK (polyether ether ketone). The supports, assembly holders, and fixtures described herein provide accurate placement of the components to one another without contacting certain parts and, for the fixtures used during bonding steps, to hold the components in contact with one another while applying no compressive force on the components are while applying a controlled amount of compressive force while avoiding causing the components being bonded bonding to the fixture(s). PTFE (e.g., TEFLON) is a material that does not undergo surface activation under plasma exposure and thereby avoids bonding to the silicone parts being treated.

Fixtures and holders used in bonding steps avoid bonding to the silicone parts being treated. Fixture materials that oxidize under plasma exposure and/or undergo surface activation are likely to adhere to the activated silicone parts. This can be avoided by selecting a material that does not oxidize or undergo surface activation (e.g., fluoropolymers such as PTFE). An alternate approach is to modify the surfaces of the fixture coming into contact with the silicone parts being bonded that are made of a potentially reactive material such as modifying with an inert coating or laser texturing. One surface can be a surface of a fixture lid and a second surface can be a surface on a fixture base. The fixture lid and fixture base can mate with one another while also holding the optical components being bonded in a desired arrangement relative to one another. The fixtures are described in more detail below. One example of such an inert coating is electroless nickel PTFE coating. Ultrashort laser texturing can be used to modify the surface energy of the areas of the fixture that contact the silicone and reduce overall contact area that can bond to the activated silicone surfaces. Examples of such patterns that can be applied for this purpose are cross-hatch, microdomes, micropillars, and microdimples.

Again with respect to FIG. 4A, the molded lens capsule 105 and posterior lens structure 110 are prepared for plasma bonding. The lens capsule 105 and/or posterior lens structure 110 can be molded as a unitary piece and plasma bonded together or may themselves be molded in two or more pieces and plasma bonded together before the lens capsule 105 and posterior lens structure 110 are then plasma bonded together.

The lens components are all prepared for plasma exposure by cleaning with an ethanol solution and dried for a period of time in a drying oven (Thermo Electron LED GmbH UT6P). To allow optimal bonding strength, all component parts of the lens are allowed to achieve room temperature and be fully dry prior to placing them into the plasma chamber. A glass petri dish holding the posterior lens structure 110 on a PEEK plate and the lens capsule 105 in a PTFE assembly holder are placed for plasma treatment within the plasma chamber. Plasma conditions for MED-6755 to MED-6820 (capsule-posterior lens) bonding are listed in Table 2 below. Upon finishing the plasma process, the plasma chamber door is opened and the petri dish containing the activated posterior lens structure 110 and capsule 105 is removed and prepared for assembly and bonding. Preferably, bonding is performed between 2 minutes and preferably no more than 30 minutes after plasma exposure.

TABLE 2
Example Plasma Conditions

| Vacuum pump operation | 1-3 | minutes |
|---|---|---|
| Pressure | 0.20-0.30 | mbar |
| Gas supply | 1-3 minute | |
| | 100% O₂ | |
| Plasma process | 20-40 | seconds |
| Venting | 1 | minute |

| | |
|---|---|
| Plasma process power | 270-300 W |
| | (90%-100%) |

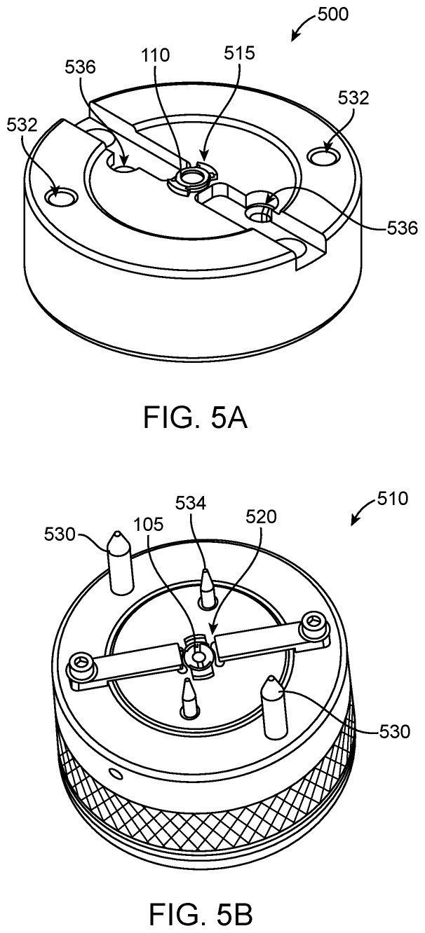

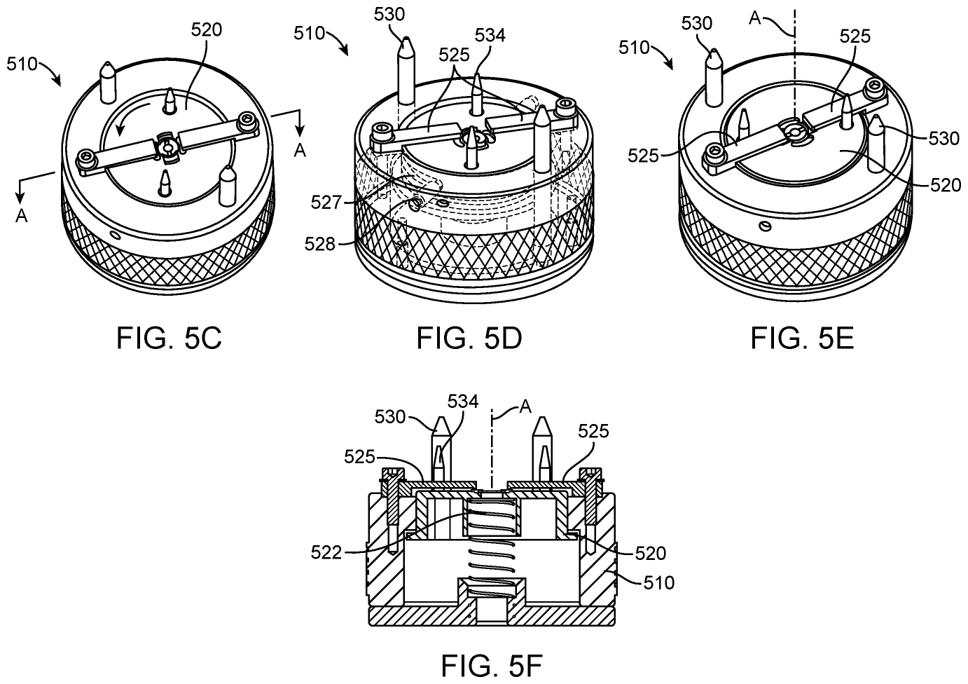

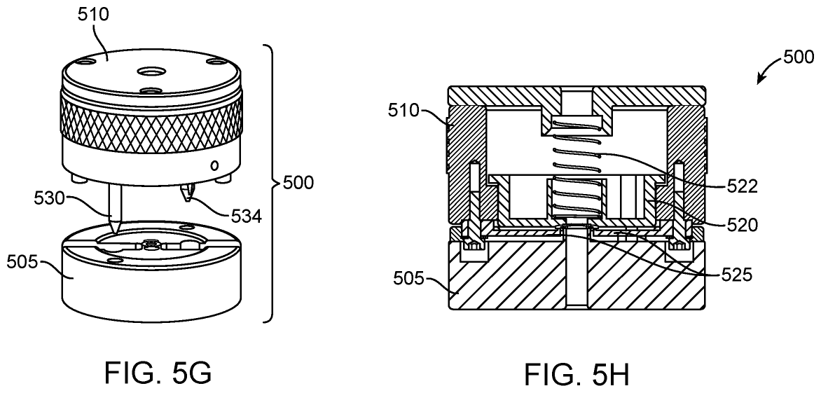

The surfaces of the activated capsule 105 are treated with alcohol prior to assembling the components, which can be assembled manually or using an assembly fixture. The activated posterior lens structure 110 can be grasped using forceps by the rib edges 116 and inverted so the convex side of the posterior lens structure 110 is facing upwards and centered over the capsule 105. The activated posterior lens structure 110 can be pressed gently with forceps on the center and the rib edges 116 near the capsule lip 117 (see FIG. 2E). Alternatively, the components can be assembled using fixtures, which will be described in more detail below with regard to FIGS. 5A-5H. Briefly, the capsule and posterior lens can then be placed inside of an aluminum assembly fixture to achieve accurate position between the surfaces that will be bonded together. The fixture allows the desired force for bonding to be applied without causing deformation. The average compression on the capsule-posterior lens assembly is between 30 and 100 grams of force. The assembly fixture 500 is placed on the petri dish in a drying oven for a period of time and then removed to cool down at room temperature.

The fixture can be used to provide accurate placement of the lens components relative to one another without needing to manually touch the components to position them relative to one another as described above. FIGS. 5A-5B show an implementation of a plasma assembly fixture 500 for bonding the activated anterior lens capsule 105 to the activated posterior lens 115. The fixture 500 can include a base 505 and a lid 510 that are configured to mate with one another. The base 505 includes a stage 515 for holding a first component such as the posterior lens structure 110. The lid 510 can also include a stage 520 for holding a second component such as the anterior capsule 105. FIGS. 5C-5E show the stage 520 of the lid 510 is configured to be rotated in order to lock the second component in place within the lid 510. Locking the second component within the lid 510 allows for the lid 510 to be inverted so as to mate it with the base 505 without the component within the lid 510 slipping or falling out. The cross-section of FIG. 5F shows the stage 520 of the lid 510 as a spring-loaded element that upon depressing the stage 520 further into the lid 510 against a spring 522, the stage 520 rotates a pre-defined distance around the central axis A of the lid 510. The rotation of the stage 520 (and thus, the second component positioned on the stage 520) relative to the rest of the lid 510 places portions of the second component into locked arrangement with a pair of fixation arms 525 on the lid 510. This locks the second component relative to the lid 510 so that it is unable to move out from the stage 520. The rotation of the stage 520 can be according to a helical groove 527 and pin 528 to achieve a pre-defined movement (see FIG. 5D). The component can be placed on the stage 520 while the stage 520 is in a first unlocked configuration. The stage 520 in the unlocked configuration is positioned so that the location of the component is rotated away from the fixation arms 525 such as by about 90 degrees. To actuate the stage 520 into the locked configuration, the stage 520 can be depressed and rotated a distance around the central axis A. As the stage 520 rotates and the pin 538 rides in the helical groove 527. When in the locked configuration, the component on the stage is engaged by each of the fixation arms and prevented from moving relative to the stage 520. A user can invert the lid 510 for mating with the base 505 when the stage is in the locked configuration without the component falling loose. The lid 510 can be actuated from the unlocked configuration to the locked configuration with the help of one or more pins 534 projecting upward away from the stage 520. The pins 534 can also help to guide the mating between the base 505 and the lid 510, which is described below.

FIGS. 5G-5H show the lid 510 mates with the base 505 aided by one or more gross positioning pins 530 in addition to the one or more fine adjustment pins 534. The gross positioning pins 530 can insert through corresponding bores 532 to achieve mating between the lid 510 and the base 505. The implementation shown in FIG. 5G shows the gross positioning pins 530 projecting from the lid 510 and the corresponding bores 532 within the base 505 although the opposite is considered as well. The one or more fine adjustment pins 534 of the lid 510 are sized to insert through corresponding bores 536. The pins 534 of the lid 510 aligns with the bores 536 in the base 505 once the stage 520 has been rotated to the locked configuration. The gross positioning pins 530 can be longer than one or more fine adjustment pins 534 so that the gross positioning pins 530 align the lid 510 and the base 505 initially followed by the fine adjustment pin 534 to minimize the inadvertent contact with the components being bonded together. The fixation arms 525 can also aid in mating of the lid 510 to the base 505. The base 505 can include two channels in its upper surface sized and shaped to receive the external surface of the fixation arms 525. Upon mating the lid 510 with the base 505, the posterior lens within the base 505 is pushed into the capsule within the lid 510 to ensure the portions of the two components to be bonded together are fully in contact with one another. The lid 510 is attached over the first component in the base 505 and a force acting on the lens is controlled by the properties of the spring 522 under the stage 520 of the lid 510. The spring 522 inside the lid 510 urges the stage 520 and the component fixed to the stage 520 downward toward the component in the base 505. Thus, the load applied onto the two components can be controlled by the spring 522. The spring 522 can have 0.1-1.0 kg force (1-10N). FIG. 5H shows the assembly in cross-section. The spring 522 is located above the stage 520 urging it downward towards the component fixed within the lid 510 by the arms 525. The component within the lid 510 is urged downwards towards the component within the base 505. The length of the pins 530, 534 is shorter than the depth of their respective bores 532, 536. Similarly, the thickness of the fixation arms 525 is less than the depth of the channel in the base 505. Thus, the components are urged together only by the force of the spring 522 and not by the weight of the lid 510.

Again with respect to FIGS. 4A-4B, following inspection of the bonded capsule-posterior lens assembly, the assembly is once again cleaned and prepared for plasma exposure. The capsule-posterior lens assembly is placed inside an assembly holder for bonding with the wing. The cleaning and drying is performed as described above. Once the component parts are dry, the capsule-posterior lens assembly in the fixture and the wing on its PEEK plate are placed on a petri dish and within the plasma chamber for plasma treatment. Example plasma conditions for MED-6755 and MED-6820 to MED-4870 (assembled capsule-Posterior Lens to Wing) bonding are listed in Table 2 above. Upon finishing the plasma process, the plasma chamber door is opened and the activated capsule-posterior lens assembly and wing are removed and prepared for assembly and bonding.

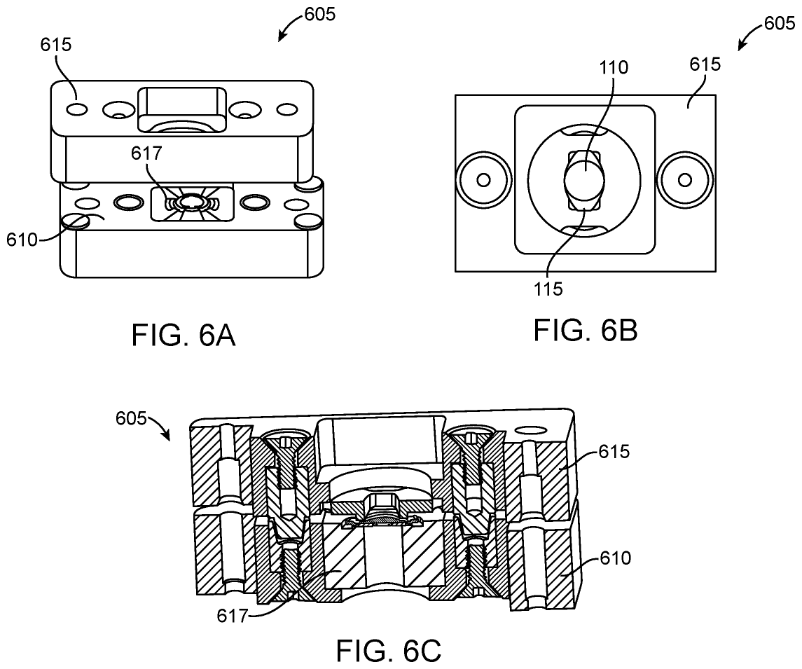

Prior to assembling the wing, all surfaces are treated with alcohol and the components assembled. The components can be assembled manually or using a fixture as described elsewhere herein. The capsule-posterior lens assembly can be placed inside an assembly holder 605 so that the wing can be bonded to the capsule-posterior lens assembly (see FIGS. 6A-6C). The assembly holder 605 can include a base 610, a cover 615, and a holder 617 such as an ULTEM ring as described elsewhere herein. The capsule-posterior lens assembly can be placed over the holder 617 and the wings placed on top. The wings can be placed manually. For example, one side of the wing can be lifted using forceps so that the wing is parallel to the holder 617. The upraised circle of the wing is placed on the indentation of the posterior lens and centered to align the wing on the capsule-posterior lens assembly. The wing covers the sealing area between the capsule-posterior lens assembly. The wing is aligned to meet the sealing area of the capsule-posterior at all four points. The wing is aligned with the side membrane area. The holder 617 can then be placed inside the assembly holder 605 and the cover 615 can be closed. The cover 615 and base 610 of the assembly holder 605 place the components between them under an amount of compressive force, for example, between 30-100 gram force compression. A petri dish containing the assembly holder 605 is placed in a drying oven for baking before being removed and allowed to cool at room temperature. Once cooled, the parts may be inspected for proper bonding.

After plasma bonding of the lens is complete, the internal chamber is sealed so that it may be filled with silicone oil. Sealing between the capsule and the posterior lens can be performed in two steps. Two out of four sealing gaps between the capsule 105 and posterior lens structure 110 are sealed. The assembled capsule 105 with the anterior optic 107 facing upwards is placed on a clean PEEK plate. The dispenser set to 40-80 psi on “purge” and dispenser syringe tipped with a 30G tip. The tip is inserted down in the gap between the capsule walls and the posterior lens structure 110. The entire gap between the capsule 105 and posterior lens structure 110 can be sealed with high temperature cure silicone adhesive such as MED2-4213. The sealing material is not used for bonding inside the optical surfaces because it is not transparent. The assembled AIOL is placed in the vertical holder to avoid leakage of the MED2-4213 when in the horizontal position. Excess MED2-4213 is wiped clean and the assembled lens placed in venting oven at 150° C. for 15 minutes. Once the assembled lens is fully sealed it is ready for oil filling, which is described in more detail in U.S. patent application Ser. No. 17/970,131, filed Oct. 20, 2022, which is incorporated by reference herein.

The IOLs described herein are preferably formed of materials configured for small incision implantation. The solid optical components of the lens can have elastomeric characteristics and can be made of soft silicone polymers that are optically clear, biocompatible, and in certain circumstances flexible having a sufficiently low Young's modulus to allow for the lens body to change its degree of curvature during accommodation. It should be appreciated that some solid optical components have a different Young's modulus than other solid optical components to provide different function to the lens (e.g. outward bowing of the anterior optic 107). Suitable materials for the solid optical component of the lens can include, but are not limited to silicone (e.g., alkyl siloxanes, phenyl siloxanes, fluorinated siloxanes, combinations/copolymers thereof), acrylic (e.g., alkyl acrylates, fluoroacrylates, phenyl acrylate, combinations/copolymers thereof), urethanes, elastomers, plastics, combinations thereof, etc. In aspects, the solid optical component of the lens is formed of a silicone elastomer, as described herein. The solid optical component can be formed of one or a combination of the materials described herein in which the liquid optical material described herein is fully encapsulated by the solid optical component. The solid optical component of a lens may include one or more regions that are configured to be in contact with and/or contain the liquid optical material. The liquid optical materials described herein can be specially formulated relative to the material of the solid optical component to mitigate lens instability and optimize optical quality. The liquid optical materials, sometimes referred to herein as an optical fluid, can include any of a variety of copolymers, including fluorosilicone copolymers and other liquid optical materials as described in PCT Publication No. WO 2021/257518, filed Jun. 15, 2021, which is incorporated by reference herein in its entirety.

Again with respect to FIGS. 1A-1C, the lens 100 include solid optical components and liquid optical material. The solid optical component can include anterior lens capsule 105 having an anterior optic 107 and the posterior lens structure 110 having a posterior optic 108. The sealed, fixed volume fluid chamber 113 can contain a fixed volume of the liquid optical material. The anterior optic 107 can include a central, dynamic zone surrounded by a static anterior optical portion at a periphery of the anterior optic. The central, dynamic zone is configured to undergo a shape change whereas the portion near the periphery can be configured to resist or not to undergo a shape change. The posterior lens structure 110 may be optically clear and provide support function without affective the optics of the lens 100. As such, the posterior optic 108 of the posterior lens structure 110 can have zero power and can form a posterior support to the lens. The posterior optic 108 of the posterior lens structure 110 can also have optical power up to about ±30 D.

The equator region of the anterior lens capsule 105 can include at least one peripheral membrane 109 that is designed to be compressed or otherwise movable along with the force transfer arms 111. Movements of the force translation arm 111 causes movements of the peripheral membrane 109 thereby deforming the liquid optical material in the fluid chamber 113 to cause a change in the shape of the anterior optic 107. As discussed above, any of a variety of the lens components may be molded together as a unitary piece or may be plasma bonded together.

The peripheral membrane 109 is coupled to or molded integral with a respective force translation arm 111. The one or more force translation arms 111 are configured to harness movements of one or more of the ciliary structures such that they are bi-directionally movable relative to the lens to effect accommodative shape change of the lens. For example, and without limiting this disclosure to any particular theory or mode of operation, the ciliary muscle is a substantially annular structure or sphincter. In natural circumstances, when the eye is viewing an object at a far distance, the ciliary muscle within the ciliary body relaxes and the inside diameter of the ciliary muscle gets larger. The ciliary processes pull on the zonules, which in turn pull on the lens capsule around its equator. This causes a natural lens to flatten or to become less convex, which is called disaccommodation. During accommodation, the ciliary muscle contracts and the inside diameter of the ring formed by the (ciliary ring diameter) ciliary muscle gets smaller. The ciliary processes release the tension on the zonules such that a natural lens will spring back into its natural, more convex shape and the eye can focus at near distances. This inward/anterior movement of the ciliary muscle (or one or more ciliary structures) can be harnessed by the force translation arms 111 to cause a shape change in the lens.

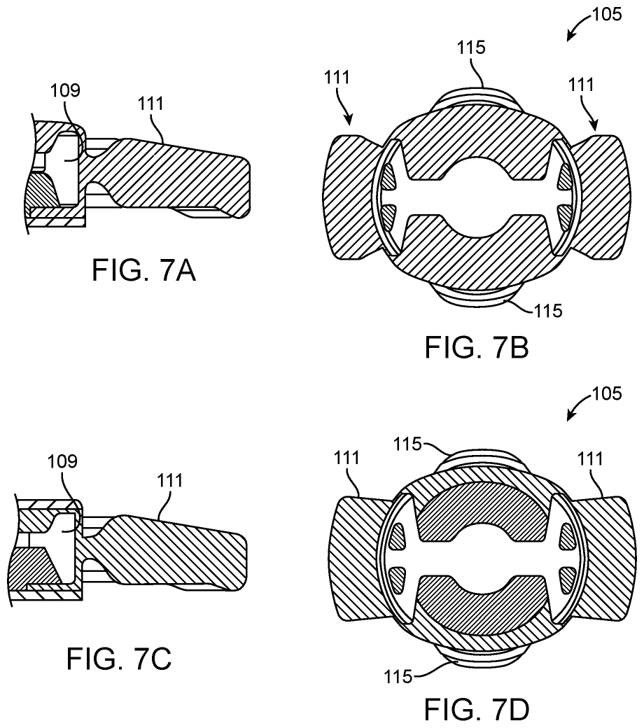

Again with respect to FIGS. 1A-1C and also with respect to FIGS. 7A-7E, the lens 100 can include preferably two force transfer arms 111 that are configured to move back and forth relative to the central visual axis of the lens to cause the dioptric changes of the lens. The lenses described herein are particularly suited to harness the movements of the ciliary body applied directly onto the force transfer arms 111 positioned against the ciliary structures into shape change of the lens. The force transfer arms 111 are configured to harness and translate forces applied by the ciliary structures into the shape changes of the movable parts of the lens body as described above. Each force transfer arm 111 can include an outer, contact portion and an inner region operatively coupled to a perimeter or equator region of the lens. As discussed above, the force transfer arms 111 can be molded integral with the anterior lens capsule 105 at the location of the peripheral membranes 109 such that the arms 111 and the membranes 109 move in concert with one another.

The force transfer arms 111 of the lenses described herein are designed to contact ciliary tissues providing substantially non-circular outer perimeter surface. Thus, the area of contact between the lens and the surrounding tissues is far less than, for example, lenses designed to be fully implanted within the capsular bag. Capsular bag lenses generally have 360 degree contact with the bag to help support the structure of the bag and maintain distance between the anterior and posterior segments of the bag. Each force transfer arm 111 of the lenses described herein can have between about 30 degree up to about 120 degree contact with the ciliary tissues. For a lens with two force transfer arms 111, this results in between about 60 degree up to about 240 degree contact between the lens and the ciliary tissues. In some implementations, each force transfer arm 111 of the lens has about 90 degree contact with the ciliary tissues providing only about 180 degree contact between the lens as a whole and the surrounding ciliary tissue. The outer contact portion 135 of the force transfer arms 111 can provide contact with the surrounding ciliary tissues that is less than about 240 degrees, about 210 degrees, about 180 degrees, about 150 degrees, about 120 degrees, about 90 degrees, down to a minimum of about 60 degrees of outer contact. The force transfer arms 111 together can have a minimum contact along an arc of 2.5 mm and a maximum contact along an arc of about 6 mm based on a ciliary process diameter of about 10.5 mm, such that the contact made by the force transfer arms 111 as a whole can be about one third of the ciliary process.

The overall shape of the force transfer arm 111 along an anterior plane of the IOL can be tapered from an outer perimeter to where the force transfer arm 111 meets the lens capsule perimeter so that the inner region of the force transfer arms 111 are narrower than their outer perimeters near where the force transfer arm 111 contacts the ciliary body (see FIGS. 7A-7B). In another implementation, the force transfer arm 111 does not taper or tapers only slightly from its outer perimeter to the inner region so that the width of the force transfer arm 111 near the lens capsule perimeter is only slight less than the width of the force transfer arm 111 at its outer perimeter (see FIGS. 7C-7D). The outer perimeter width of the force transfer arm can be about 0.25 mm to about 0.75 mm wider (about 5% up to about 18% wider) than the inner region width.

The liquid optical material contained within the fluid chamber 113 can be a non-compressible liquid optical material and the volume of the fluid chamber 113 can be substantially identical to the volume of liquid optical material. As such, the liquid optical material contained within the chamber 113 does not cause significant outward bowing of either the anterior optic 107 or the peripheral membrane 109 in the resting state when no substantial outside forces are applied to the lens 100. In aspects, the fluid chamber 113 can be slightly overfilled with liquid optical material such that the anterior optic 107 has some outward bowing at rest. A small degree of resting outward bowing in the anterior optic 107 can reduce optical artifacts in the lens. However, no matter how much resting outward bowing is present in the anterior optic 107, it can still undergo additional outward bowing upon application of compressive forces on the peripheral membrane 109 to provide accommodation. Because the liquid optical material in the fluid chamber 113 is non-compressible its shape deforms along with the shape of the chamber 113. Deformation of the chamber 113 in one location (e.g. micrometer inward movements of the peripheral membrane 109) causes the non-compressible liquid optical material contained within the fixed-volume fluid chamber 113 to press against the inner-facing surface of the anterior optic 107 to create sufficient accommodating change.

The wings 115 provide stabilization of the lens within the eye. The wings 115 are configured to maintain alignment of the optics and resist movement of the lens once implanted and undergoing shape changes. The wings 115 preferably do not cause accommodation of the lens. Thus, the force transfer arms 111 are only involved in the accommodation of the lens whereas the wings 115 provide fixation, centering, stabilization, and/or hold the lens in position within the eye. As described above, the wings 115 can be molded separately and plasma bonded to the lens on a posterior region of the device so that it can provide stabilization and engagement with a portion of the capsular bag, such as with the anterior capsule.

The devices and systems described herein can incorporate any of a variety of features. Elements or features of one implementation of a device and system described herein can be incorporated alternatively or in combination with elements or features of another implementation of a device and system described herein as well as the various implants and features described in. For the sake of brevity, explicit descriptions of each of those combinations may be omitted although the various combinations are to be considered herein. Provided are some representative descriptions of how the various devices may be manufactured, however, for the sake of brevity explicit descriptions of each method with respect to each implant or system may be omitted.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. A method of manufacturing an accommodating intraocular lens, the method comprising:
molding an optical component having at least a central portion formed of an optically clear silicone material;
molding a second component, the second component comprising a silicone material;
activating surfaces of the optical component and surfaces of the second component in a plasma chamber; and
covalently bonding together without adhesive the optical component and the second component to form a lens capsule having an internal chamber.

2. The method of claim 1, further comprising applying a vacuum pressure within the plasma chamber during the activating.

3. The method of claim 2, wherein the vacuum pressure is between 0.01 mbar and 1 mbar.

4. The method of claim 1, wherein a process power of the plasma chamber during the activating is between 270-300 W.

5. The method of claim 1, further comprising supplying a gas to the plasma chamber during the activating.

6. The method of claim 5, wherein the gas is Argon, atmospheric air, or oxygen.

7. The method of claim 5, wherein the gas is supplied with or without applying vacuum pressure to the plasma chamber.

8. The method of claim 5, wherein the gas comprises 70%-100% oxygen.

9. The method of claim 1, further comprising holding the optical component and the second component within a fixture during the covalently bonding.

10. The method of claim 9, wherein the fixture is designed to apply an amount of compression between the surfaces of the optical component and the surfaces of the second component.

11. The method of claim 10, wherein the amount of compression is between 30-100 gf.

12. The method of claim 9, wherein the fixture is designed to hold the optical component and the second component in contact with one another without applying compressive force on the optical component and the second component.

13. The method of claim 9, wherein the fixture is formed of a material that does not bond to the optical component or the second component during covalently bonding.

14. The method of claim 13, wherein the fixture is a high-heat resistant material.

15. The method of claim 13, wherein the fixture is polytetrafluoroethylene (PTFE).

16. The method of claim 9, wherein the fixture comprises a first surface contacting the optical component and a second surface contacting the second component.

17. The method of claim 16, wherein the first surface and the second surface are modified to prevent bonding to optical component and the second component, respectively.

18. The method of claim 17, wherein the first surface and the second surface are modified by coating or by laser texturing.

19. The method of claim 16, wherein the first surface is on a fixture lid and the second surface is on a fixture base, wherein the fixture lid and the fixture base are configured to mate with one another while also holding the optical component and the second component relative to one another.

20. The method of claim 19, wherein the fixture lid comprises a pin and the fixture base comprises a helical groove, wherein the pin rides within the helical groove as the fixture lid is locked with the fixture base.

21.-42. (canceled)
